(19) United States
(12) Patent Application Publication
(10) Pub. No.: US 2013/0025591 A1
(43) Pub. Date: Jan. 31, 2013

(54) Ventilator with Integral Oxygen Generator

(71) Applicants: Robert L. Clark, Bellevue, WA (US); Jerry Luedke, Maple Valley, WA (US); Scott Thielman, Seattle, WA (US); Richard Taylor, Fall City, WA (US); Blake Stancik, Mukilteo, WA (US); Alan Widmer, Bellevue, WA (US); Brian J. Doll, Sammamish, WA (US)

(72) Inventors: Robert L. Clark, Bellevue, WA (US); Jerry Luedke, Maple Valley, WA (US); Scott Thielman, Seattle, WA (US); Richard Taylor, Fall City, WA (US); Blake Stancik, Mukilteo, WA (US); Alan Widmer, Bellevue, WA (US); Brian J. Doll, Sammamish, WA (US)

(73) Assignee: Wyngate Medical Technologies, Inc., Bellevue, WA (US)

(21) Appl. No.: 13/487,160

(22) Filed: Jun. 2, 2012

Related U.S. Application Data
(60) Provisional application No. 61/493,269, filed on Jun. 3, 2011.

(51) Int. Cl. A61M 16/10

(52) U.S. Cl. 128/202.26

(57) ABSTRACT

A method and apparatus provide respiratory ventilation support to a patient by concentrating oxygen with an oxygen generator comprising a nitrogen adsorbing molecular sieve and mixing and supplying the concentrated oxygen and ambient air. The method and apparatus determine parameters for providing respiratory ventilation support to the patient based on an indication of the size of the patient provided by an operator of the apparatus.

### CROSS REFERENCE TO RELATED APPLICATIONS

This application claims the benefit of U.S. Provisional Application No. 61/493,269, filed Jun. 3, 2011, which is incorporated by reference in its entirety.

### FIELD OF THE INVENTION

The present invention relates to the field of respiratory ventilators and more particularly to mobile ventilators capable of generating oxygen.

### BACKGROUND

Mass casualty events, such as pandemics, regional natural disasters, terrorist attacks, chemical or nuclear disasters, may result in a large population of patients with Acute Respiratory Distress Syndrome (ARDS), which is a condition that occurs when not enough oxygen passes from a patient's lungs into his blood or when a patient's lungs cannot remove waste carbon dioxide in his blood, which can damage organs of the body. Current treatments for a patient with ARDS typically require Intensive Care Unit (ICU) care, wherein the patients are supported by mechanical ventilation connected to supplemental oxygen tanks and power supply units. A ventilator is a machine that helps a patient breathe by blowing air (or air with increased amounts of oxygen) into his airway and lungs until the patient can effectively breathe on his own. Hospital mechanical ventilation systems are typically substantial, complicated, and expensive systems integrated with a hospital central oxygen storage system that delivers compressed oxygen to individual hospital rooms. Additionally, patients in ICU care and supported by mechanical ventilation must be supervised by highly trained respiratory specialists to ensure that the patient is ventilated properly and provided with an adequate amount of oxygen.

However, hospitals and ICUs are poorly equipped to provide patient care in case of mass casualty events. For example, mass casualty events are typically associated with severe shortages in ICU beds available for a large patient population that needs care and can result in damage to central oxygen storage facilities. Additionally, ventilators that meet Center for Disease Control (CDC) requirements are also typically in short supply to meet the needs of patients in case of mass casualty events because ventilators occupy a large amount of space on an ICU floor. Similarly, compressed air and oxygen supplies are also generally in short supply as it may be expensive or impractical for an ICU to carry enough to meet the needs of patients in the event of a mass casualty. Finally, well trained respiratory specialists needed to treat the many patients in need during a mass casualty event may not be readily available. As a result, ICU units and ventilators are ill equipped to provide care or respiratory support to patients, particularly during a mass casualty event.

Some ventilation support systems are portable and have a capability to provide oxygen to a patient in a variety of different environments. However, such portable ventilators still need to be operated and administered by trained professionals. Additionally, such portable ventilators have a small oxygen tank or oxygen (O₂) cylinders which provide ventilation support for a small period of time. Portable ventilators which generate oxygen do not generate oxygen at a high enough rate or at sufficient purity for ventilating seriously ill patients. Therefore, such portable ventilators provide limited functionality and a limited time of usage.

### SUMMARY

A method and apparatus provide respiratory ventilation support to a patient. The apparatus comprises an oxygen generator comprising a nitrogen adsorbing molecular sieve for concentrating a supply of oxygen. A ventilation apparatus, such as a mechanical ventilator, mixes the oxygen with ambient air and supplies the mixed oxygen and air to the patient. The apparatus comprises a memory configured to be executed by a processor. The memory comprises instructions for determining ventilation parameters based on input from an operator, such as input indicating patient size, received from a user interface.

In some embodiments, the apparatus adjusts the rate of generation of oxygen by the oxygen generator based on the rate of oxygen required to be provided to the patient as determined in the ventilation parameters.

In some embodiments, oxygen is stored in a carbon molecular sieve prior to being mixed with ambient air for provision to a patient.

In some embodiments, the differential pressure between the patient's lungs and that provided to the patient is monitored to estimate the flow of breathing gas to patient. Optionally a ratio of concentrated oxygen to ambient air is adjusted based on the flow estimate.

### BRIEF DESCRIPTION OF THE DRAWINGS

The teachings of the embodiments of the present invention can be readily understood by considering the following detailed description in conjunction with the accompanying drawings.

FIG. 1 is a high-level block diagram of a system including a ventilation support system, in accordance with an embodiment of the invention.

FIG. 2 is a high-level block diagram of an oxygen generator housed in a ventilation support system, in accordance with an embodiment of the invention.

FIG. 3 is a high-level block diagram of a ventilator housed in a ventilation support system, in accordance with an embodiment of the invention.

FIG. 4 is a process diagram for providing ventilation support to a patient, in accordance with an embodiment of the invention.

FIG. 5 illustrates the ventilation support system in greater detail, in accordance with an embodiment of the invention.

FIG. 6 illustrates an example user interface displayed by the ventilation support system, in accordance with an embodiment of the invention.

FIG. 7A illustrates a ventilation support system, in accordance with an embodiment of the invention.

FIG. 7B illustrates a ventilation support system being transported by a user, in accordance with an embodiment of the invention.

FIG. 7C illustrates components housed within a ventilation support system, in accordance with an embodiment of the invention.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

Embodiments are now described with reference to the figures where like reference numbers indicate identical or functionally similar elements. Also in the figures, the left most digit of each reference number corresponds to the figure in which the reference number is first used.

Reference in the specification to “one embodiment” or to “an embodiment” means that a particular feature, structure, or characteristic described in connection with the embodiments is included in at least one embodiment. The appearances of the phrase “in one embodiment” or “an embodiment” in various places in the specification are not necessarily all referring to the same embodiment.

In addition, the language used in the specification has been principally selected for readability and instructional purposes, and may not have been selected to delineate or circumscribe the inventive subject matter. Accordingly, the disclosure of the embodiments is intended to be illustrative, but not limiting, of the scope of the embodiments, which are set forth in the claims.

### System Overview

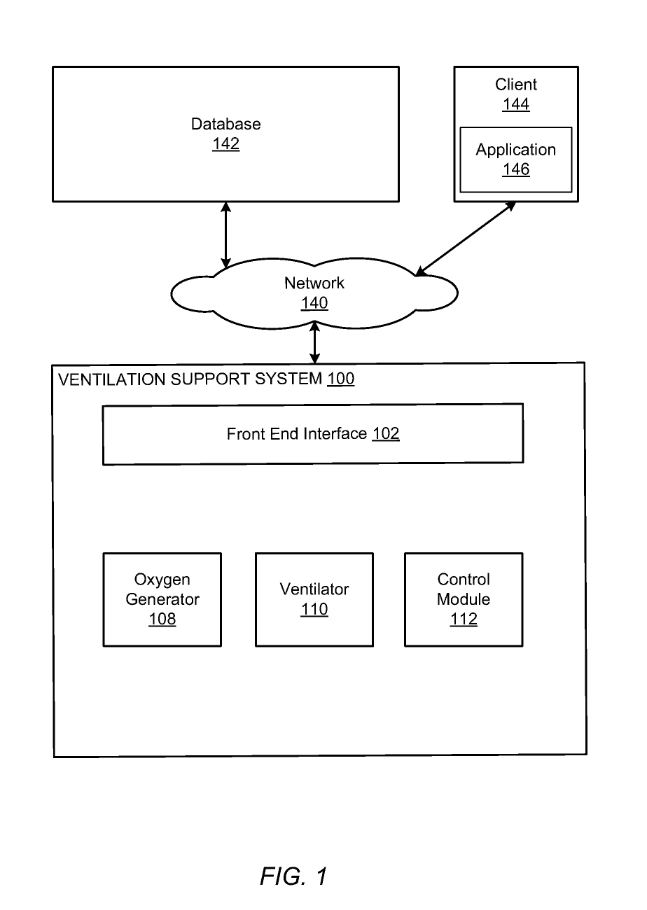

FIG. 1 is an illustration of a high-level block diagram of a system including a ventilation support system 100, in accordance with an embodiment of the invention. As shown in FIG. 1, a ventilation support system 100 comprises a front end interface 102, an oxygen generator 108, a ventilator 110 and a control module 112.

Most generally, the ventilation support system 100 represents a mobile ventilation unit that is enabled to generate its own supply of oxygen. In addition, with the other components consolidated within the ventilation support system 100, the device is self contained. The ventilation support system 100 represents a device that is portable, rugged, cost effective and easy to deploy and use in several different settings, including a hospital.

At a high level, the ventilation support system 100 receives a user input from the front end interface 102. In one embodiment, the user input includes a selection most closely matching the patient's age and size. Responsive to receiving this selection, the control module 112 determines the oxygen requirements for the patient and the ventilation support system 100 draws air from the ambient room air and provides it to the oxygen generator 108 which generates the oxygen having the required purity. It is noted that the oxygen generator 108 may also be referred to as an oxygen concentrator in the specification. The oxygen generator 108 filters, chills and pressurizes the air drawn by the ventilation support system 100 creating compressed air. In addition, the oxygen generator 108 generates an oxygen supply to ventilate a patient. In one embodiment, the oxygen generator uses a Pressure Swing Adsorption (PSA) system to capture nitrogen from the ambient air drawn in by the ventilation support system 100 using an adsorbent. This results in oxygen-enriched air which is the supply of oxygen for providing to a patient. The oxygen generator 108 then provides the oxygen and compressed air to the ventilator 110. The ventilator 110 regulates, meters and mixes the streams of oxygen and compressed air to provide the mixture to the patient.

The ventilation support system 100 can communicate with a database 142 and/or a client device 144 via a network 140, which is typically the internet, but can also be any network, including but not limited to any combination of LAN, a MAN, a WAN, a mobile, wired or wireless network, a private network, or a virtual private network.

The database 142 stores sensor data and patient diagnostic data collected by the ventilation support system 100. In one embodiment, the database 142 receives the data over the network 140 and stores it so that it may be accessible by one or more computing devices over wired or a wireless network.

The client device 144 is a computing device that receives sensor data and patient diagnostic data from the ventilation support system 100 over the network 140. In one embodiment, an application 146, such as a browser, may be used to access and display the received data on the client device 144. In other embodiments, the client device 144 may be a mobile computing device, such as a laptop, a cell-phone, a tablet computer, etc., such that another user or a medical professional may view patient diagnostic data remotely as a user administers the ventilation to a patient via the ventilation support system 100. In one embodiment, the ventilation support system 100 sends a video feed of the ventilation administration to the patient to the client device 144 such that a viewer or a medical professional may oversee or provide instructions on proper administration techniques. In other embodiments, the client device 144 may receive additional information from the ventilation support system 100 to help administer ventilation support to a user.

The control module 112 comprises at least one processor coupled to a chipset. Also coupled to the chipset are a memory and a storage device. The control module is optionally coupled to a display on the ventilation support system 100 via a graphics adapter. In one embodiment, the functionality of the chipset is provided by a memory controller hub and an I/O controller hub. In another embodiment, the memory is coupled directly to the processor instead of the chipset. In some embodiments, the control module 112 can be coupled to a keyboard and/or a pointing device.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the control module 112. The graphics adapter displays images and other information on the display. The network adapter couples the computer system to a local or wide area network.

The control module 112 can have different and/or other components than those described previously. In addition, the control module 112 can lack certain components. Moreover, the storage device can be local and/or remote from the control module 112 (such as embodied within a storage area network (SAN)).

As is known in the art, the control module 112 is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

### Oxygen Generator

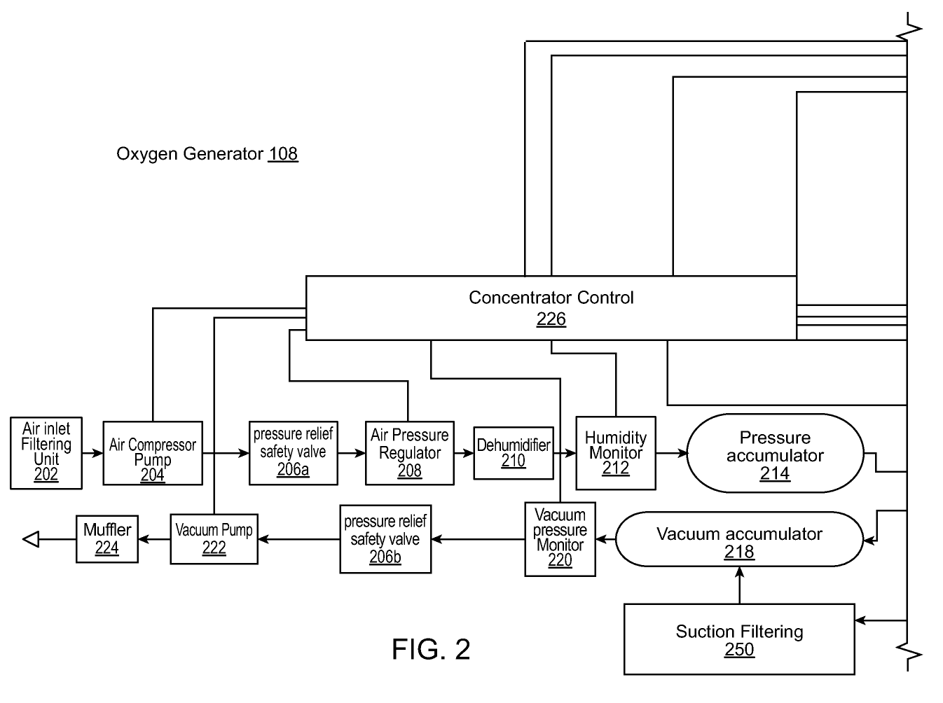

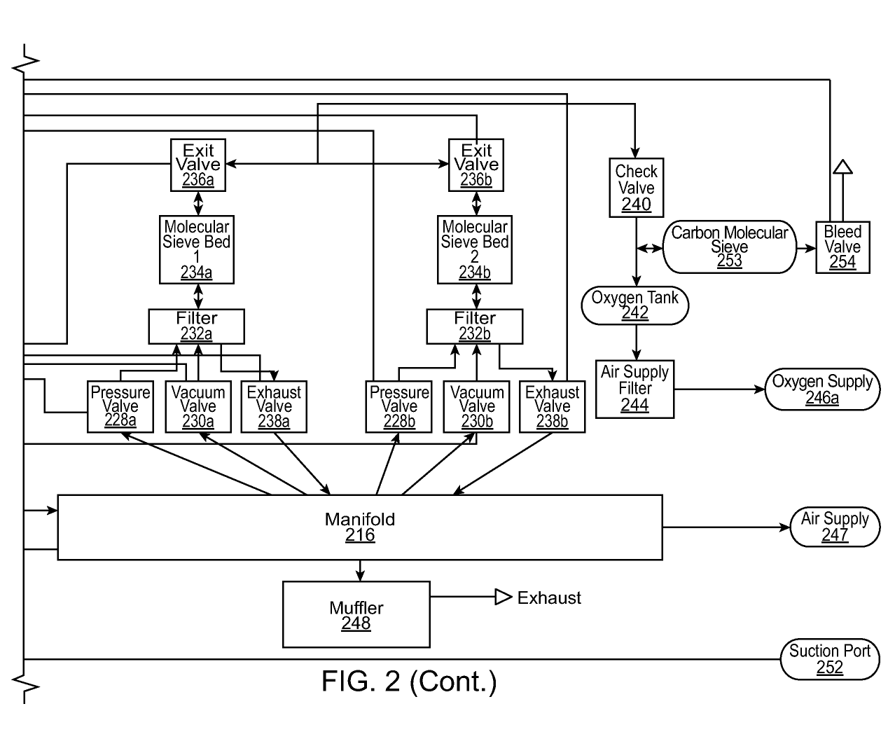

FIG. 2 is a high-level block diagram of an oxygen generator 108 housed in a ventilation support system 100, in accordance with an embodiment of the invention. In one embodiment, the oxygen generator 108 uses PSA to remove nitrogen from the air drawn by the ventilation support system 100 to provide oxygen for a patient. PSA technology separates some gas species such as nitrogen from a mixture of gases by applying pressure. Under high pressures, gases tend to be attracted to solid surfaces or adsorbed. The higher the pressure, the more gas is adsorbed; when the pressure is reduced, the gas is released, or desorbed. PSA processes can be used to separate gases in a mixture because different gases tend to be attracted to different solid surfaces more or less strongly. If a gas mixture such as air, for example, is passed under pressure through a vessel containing an adsorbent bed of zeolite that attracts nitrogen more strongly than it does oxygen, part or all of the nitrogen will stay in the bed, and the gas coming out of the vessel will be enriched in oxygen. In one embodiment, the zeolite comprises lithium. When the bed reaches the end of its capacity to adsorb nitrogen, it can be regenerated by reducing the pressure, thereby releasing the adsorbed nitrogen. It is then ready for another cycle of producing oxygen enriched air.

The oxygen generator 108 includes an air inlet filtering unit 202, an air compressor pump 204, pressure relief valves 206, an air pressure regulator 208, a dehumidifier 210, a humidity monitor 212, a pressure accumulator 214, a manifold 216, a vacuum accumulator 218, a vacuum pressure monitor 220, a vacuum pump 222, a muffler 224, a concentrator control 226, pressure valves 228, vacuum valves 230, filters 232, molecular sieve beds 234, exit valves 236, exhaust valves 238, a check valve 240, an oxygen tank 242, an air supply filter 244, oxygen supply 246, a muffler 248, suction filter 250, a suction port 252, a carbon molecular sieve 253 and a bleed valve 254.

In one embodiment, the air inlet filtering unit 202 filters ambient air drawn by the ventilation support unit 100. The air is filtered to prevent degradation of components within the ventilation support unit 100 including the air compressor pump 204. The air compressor pump 204 compresses the filtered air to supply the breathing gas needs of the ventilation support unit 100. In one embodiment, the air compressor pump 204 compresses the drawn air to a 30-40 psi pressure range. The air compressor pump 204 may include, but is not limited to a wobble compressor, a vane compressor, a scroll compressor, a twin screw compressor; driven by an AC motor, a brushless DC motor, etc. The pressure relief safety valve 206a provides a relief if the air is compressed beyond a predetermined pressure. The air pressure regulator 208 regulates the air pressure. The dehumidifier 210 removes humidity by cooling the pressurized air and allowing the moisture to condense out. The humidity monitor 212 measures the humidity in the compressed air. The pressure accumulator 214 accumulates the compressed air. In one embodiment, the pressure accumulator 214 may comprise a tank of 3 to 4 liters.

The manifold 216 directs pressure and vacuum to various components within the ventilation support unit 100. The manifold directs vacuum to a vacuum accumulator 218 to regulate pressure in the manifold 216. The vacuum pressure monitor 220 monitors the pressure in the vacuum accumulator 218 and the pressure relief safety valve 206b provides relief to the vacuum accumulator for increasing pressure depending on the pressure therein. The vacuum pump 222 pumps the vacuum out through the muffler 224 as exhaust into ambient air. The suction port 252 (normally closed or capped) allows ambient air to be drawn through a suction filter 250 and into the vacuum accumulator 218. This suction port 252 serves as a port for optionally attaching a suction collection bottle making general suction available to the operator.

The manifold 216 interfaces with the molecular sieve beds 234 to perform PSA. In one embodiment, the molecular sieve beds 234 comprise concentrator materials such as zeolites. MDX and 5XP materials marketed by UOP HONEYWELL are examples of zeolite materials commonly available and may be used with the disclosed apparatus. In one embodiment, the manifold pressurizes one molecular sieve bed and exhausts another.

The following describes the typical cycle for molecular sieve bed 1 234a. The cycle for molecular sieve bed 2 234b is the same but 180 degrees out of phase. The manifold 216 directs pressure through a rotary valve, wherein the pressure valve 228a provides the pressure to a filter 232a. The pressure is further provided to the molecular sieve bed 1 234a, wherein the molecular sieve bed 234a adsorbs nitrogen from the pressurized air. The resulting unadsorbed gas (hereinafter also referred to as ‘product gas’) is passed through the exit valves 236 and check valve 240 to an oxygen tank 242.

In some embodiments, a portion of the product gas is directed to a carbon molecular sieve 253. Carbon molecular sieve materials are available from a variety of commercial sources such as Hengye USA, CarboTech AC GmbH and Y-Carbon. Under the effect of increasing gas pressure, oxygen preferentially diffuses into the carbon molecular sieve 253 and becomes trapped there. The molecules that become entrapped no longer contribute to the gas pressure of the sieve. Thus, it is possible for a vessel filled with carbon molecular sieve to contain a larger volume of oxygen than an equally sized vessel without the carbon material. This ability to buffer the oxygen supply in a small compact vessel is advantageous for a generator system that is meant to be portable because more oxygen can be stored. In one example, if a patient suddenly needs a higher percentage of oxygen than that being generated by the oxygen generator 108, the oxygen stored in the buffer at the carbon molecular sieve 253 provides the required additional oxygen until the oxygen generator's 108 production is up to the new requirements as instructed by the control module 112. If excess nitrogen molecules, not adsorbed by the molecular sieve beds 234 begin to collect in the carbon molecular sieve 253, the bleed valve 254 is activated by the concentrator control 226 to exhaust this excess nitrogen. Use of the bleed valve 254 in conjunction with the carbon molecular sieve 253 increases the purity of the oxygen supplied to the oxygen supply 246a.

Thereafter, the manifold 216 exhausts the bed 234a via the exhaust valve 238a, allowing the compressed gas within 234a to escape to atmosphere. In some embodiments, a vacuum apparatus applies a vacuum to the molecular sieve bed 234a causing the molecular sieve bed 234a to release the adsorbed nitrogen. In one embodiment, the manifold 216 provides vacuum to the molecular sieve bed 234a and the adsorbed nitrogen is released to the manifold 216 through the filter 232a and the vacuum valve 230a. Reducing the partial pressure of nitrogen in the molecular sieve bed 234a encourages the adsorbed nitrogen to desorb and leave the system, allowing more capacity for adsorption during the next pressurization phase. This reduction in partial pressure is achieved by lowering the total pressure in the tank via exhaust and vacuum valves (238a and 230a) but also by allowing some amount of purge gas into the top of the tank flowing from 234b through 236b and into 236a. This purge gas, having a high concentration of oxygen, acts to further lower the partial pressure of nitrogen as well as helping to sweep the desorbed gas out of the sieve. In alternate embodiments, the reduction of partial pressure of nitrogen in the molecular sieve beds 234 is accomplished with exhaust valves 238 and purge without the use of a vacuum.

The filter 232 filters the gas flow out of the sieve 234a during the exhaust and vacuum phases before it re-enters the manifold 216. This prevents possible contamination of the manifold and ambient room air with zeolite dust. In some embodiments, the oxygen may be concentrated using vacuum pressure swing adsorption (VPSA), rapid pressure swing adsorption (RPSA), vacuum swing adsorption (VSA), duplex PSA and modified duplex PSA.

The air supply filter 244 traps any particulates from the molecular sieve bed 234 and carbon molecular sieve 253. This ensures that only clean safe breathing gas is supplied to the oxygen supply port 246a. The manifold 216 directs pressurized air from the pressure accumulator 214 to the air supply 247. The air supply 247 is a source of pressurized, clean, 21% oxygen content breathing gas for the subsequent ventilator stage.

In one embodiment, the oxygen generator 108 generates oxygen at a selectable rate between 0 and 20 L/min. In one embodiment, the oxygen generator 108 generates oxygen at a rate of 20 L/min or greater. In some embodiments, the oxygen generator 108 generates oxygen at a rate between 20 and 30 L/min. In some embodiments, the oxygen generator 108 generates oxygen at a rate between 20 and 25 L/min. In some embodiments, the oxygen generator 108 generates oxygen at a rate between 22 and 24 L/min.

In some embodiments, the generated oxygen is more than 90%, 91%, 92%, 93%, 94%, 95%, 96%, 97%, 98% or 99% oxygen.

### Ventilator

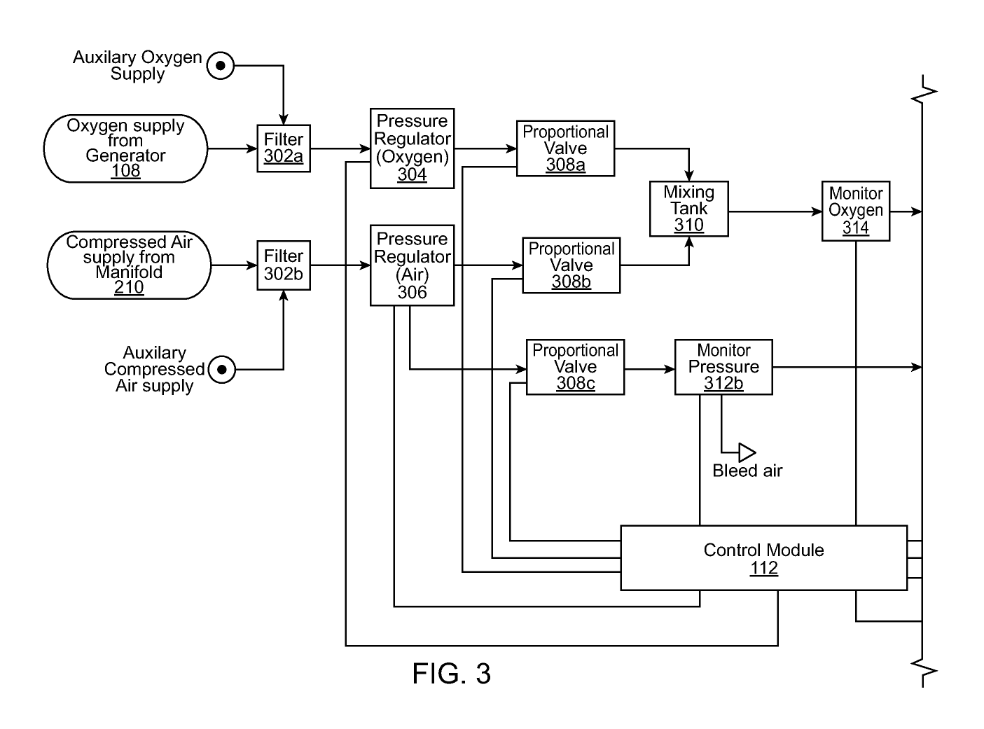

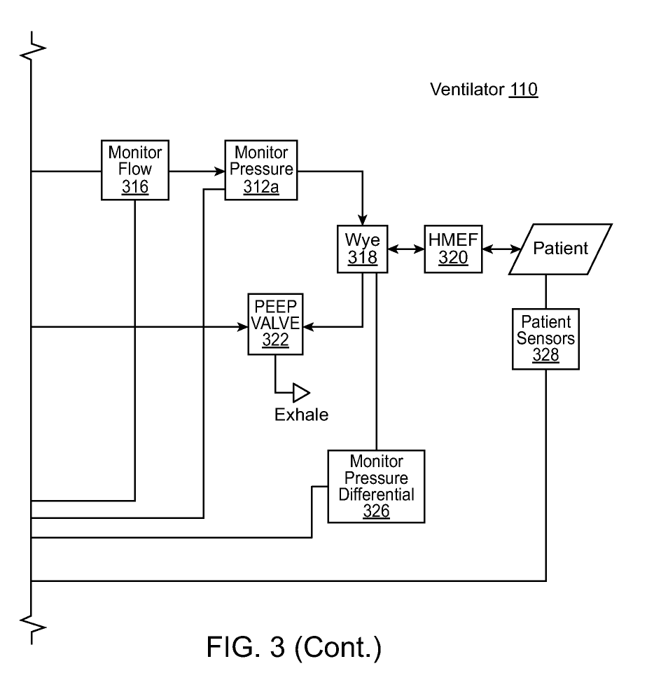

FIG. 3 is a high-level block diagram of a ventilator 110, in accordance with an embodiment of the invention. The ventilator 110 component is enabled to move breathable air in and out of lungs of a patient attached to the ventilation support unit 100. The ventilator 110 includes filters 302, an oxygen pressure regulator 304, an air pressure regulator 306, proportional valves 308, a mixing tank 310, pressure monitors 312, an oxygen monitor 314, a flow monitor 316, a wye 318, a heat and moisture exchanger and filter (HMEF) 320, a positive end-expiratory pressure (PEEP) valve 322, the control module 112, a differential pressure monitor 326, and patient sensors 328.

The ventilator 110 receives a supply of oxygen from the oxygen supply 246a within the oxygen generator 108 and a supply of compressed air from the air supply 247 within the oxygen generator 108. Additionally, the oxygen and the air can be provided by an auxiliary oxygen supply and an auxiliary compressed air supply attached to the ventilation support systems 100. The filter 302a filters the supplied oxygen and the filter 302b filters the air supply. The oxygen pressure regulator 304 regulates the oxygen pressure and passes the oxygen through a proportional valve 308a for mixing with air. Similarly, the air pressure regulator 306 the air pressure and provides the air to the proportional valve 308b to mix the air with oxygen. In one embodiment, the ventilator 110 includes pressure sensors in the place of the oxygen pressure regulator 304 and the air pressure regulator 306. In such an embodiment, the proportional valves 308 receive oxygen and air from the oxygen generator 108 at a variety of pressures and the control module 112, using the pressure information, modulates the proportional valves 308 to provide the correct flow. In yet another embodiment, a flow sensor is used instead of the oxygen pressure regulator and the air pressure regulator as illustrated in FIG. 3. The flow information allows the control module 112 to meter flow through the proportional valves to vary the flow of oxygen and air provided to the mixing tank 310.

The proportional valves 308 regulate the flow of oxygen and air permitted to mix with each other. The proportional valve 308c and monitor pressure sensor 312b are used in a control loop to set the peak end expiratory pressure (PEEP) at which the exhalation valve PEEP valve 322 opens. The oxygen monitor 314 measures the amount of oxygen in the mixed air and the flow monitor 316 measures the flow of the mixed air. The pressure monitor 312a measures the mixture's pressure.

The wye interchange 318, the PEEP valve 322 and the HMEF 320 are part of a patient assembly. The mixture is provided to the wye interchange 318 which is essentially a fork in the air mixture's path and enables the air mixture to go either to the HMEF 320 or the PEEP valve 322. The PEEP represents pressure in the patient's lungs above atmospheric pressure that exists at the end of expiration. The PEEP valve 322 prevents pressure in a patient's lung from getting too high. As such, the valve is closed during inhalation by the control module 112. The control module 112 also controls the oxygen pressure regulator 304, the air pressure regulator 306, the proportional valves 308 and receives signals from the various monitors in the ventilator 110 including the oxygen monitor 314, the flow monitor 316, the pressure monitors 312 and the differential pressure monitors 326. The HMEF 320 filters air provided to the patient. In one embodiment, the HMEF 320 takes moisture collected by the adsorbent materials and moisture exhaled by the patient and adds it to the inlet air provided to the patient. The HMEF 320 reduces the need for cleaning tubing and equipment associated with the patient assembly and protects the patient and the personnel from various microorganisms present in the breathing circuit.

The patient sensor 328 monitors the patient's breathing and the differential pressure monitor 326 monitors the difference between the patient's lung pressure and the pressure of the inlet air provided by the ventilator 110. The differential pressure monitor 326 senses the pressure difference across the wye 318. This pressure difference corresponds to a volumetric flow rate to the patient. This information is used by the control module 112 to implement various ventilation protocols. For example the control module 112 adjusts the ratio of oxygen to air if needed based on the measured differential pressure.

The patient sensors 328 can be any sensor monitoring any of a variety of patient metrics such as, but not limited to, pulse oximetery, blood gas concentrations, electro-cardiogram, blood pressure. This information is made available to the control module 112 allowing the system to report patient status to a clinician. Additionally, the control module 112 can use these metrics from the patient sensors 328 to automatically adjust the ventilator settings to increase or decrease the amount of oxygen delivered to the patient with the goal of maintaining the metrics within certain bounds.

### Providing Ventilation

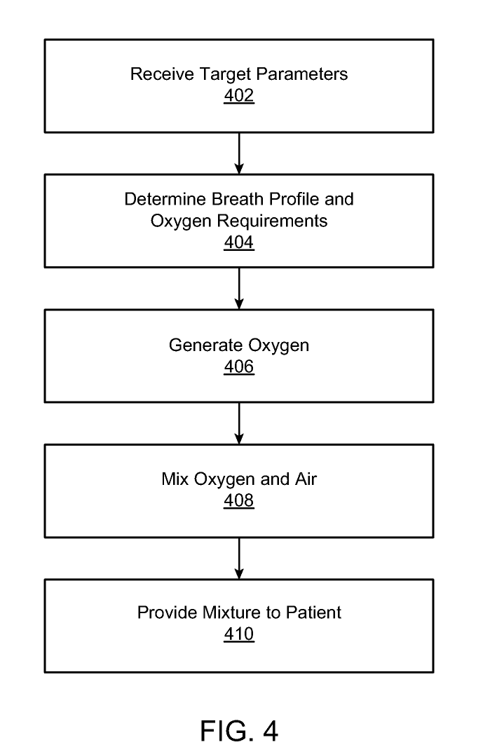

FIG. 4 illustrates a process for providing breathing air to a user in order to provide ventilation support to a user. This process can be performed using the ventilation support system 100 and is described with regard to ventilation support system 100 below, but this process can also be performed using other ventilation systems. In one embodiment, the control module 112 receives 402 a patient's target parameters, including for example, but not limited to, the user's approximate height, weight, age, medical history, condition, etc.

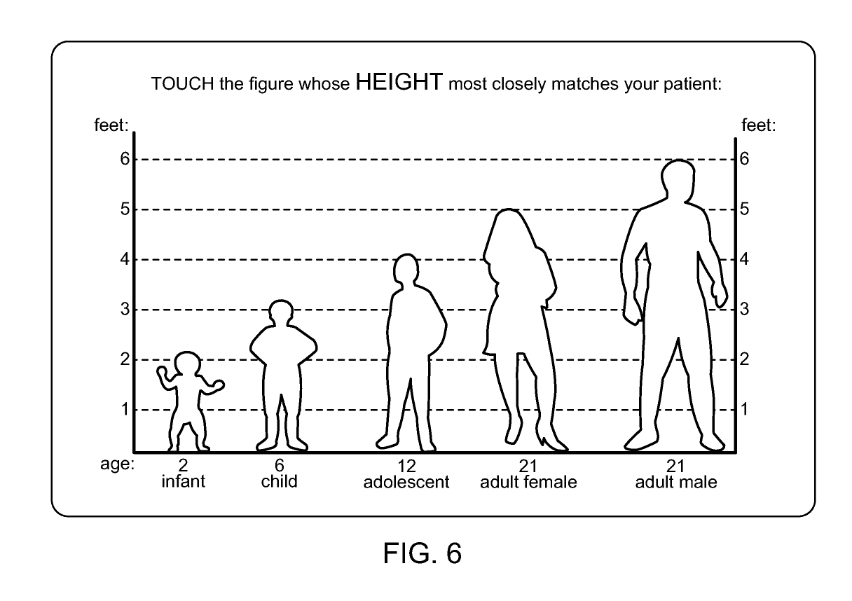

FIG. 6 is an illustration of a user interface according to one embodiment which allows for a user of the ventilation support system 100 to provide patient information to the ventilation support system 100. For example, the user may be shown an outline of humans of different ages and/or sizes wherein the user may be requested to select an outline that best matches the patients' age and/or size. Humans of different ages and/or sizes have different oxygen requirements. If the patient is an adult male, the user might select the rightmost outline in the figure labeled “adult male.” The ventilation support system 100 uses the user provided information to determine an appropriate amount of oxygen to provide to a patient.

FIG. 6 shows outlines of humans ranging from infants to adults. This can include outlines for persons of both sexes, as females may have different oxygen requirements than males. In the example presented in FIG. 6, the interface includes not only the age that each of the outlines is intended to represent (see bottom of figure, along the x-axis) but also includes the size that each outline is intended to represent (see sides, along the y-axis). Other examples of the interface may include just one of these two. In addition, a variety of other ages and sizes can be included. The user can select one of the outlines in various ways. For example, if the user interface is a touch screen, the user can simply touch the appropriate outline. As another example, the interface might include buttons or other controls associated with the different options that the user can select. The selection of different outlines provides the benefit of allowing users speaking different languages or otherwise having limited ability to understand user interfaces to easily select a visual representation that best matches the patient.

In other embodiments, other user interfaces may be provided to the user to enable the user to enter parameters about the patient. For example, instead of providing outlines of human figures, the interface could simply provide words describing the options, such as “adult male,” “female child,” “adolescent male,” etc. that a user can select or could provide numbers representing different estimated heights, weights, ages, etc. of the patient. The user interface may be designed in such a way that a lay person, or a person without medical training, can enter pertinent information about the patient.

Returning to FIG. 4, the control module 112 determines 404 the patient's oxygen requirements based on the input information provided by the user. For example, the control module 112 may calculate a patient's lung capacity based on statistical data and the patient information input by the user. An estimate of lung capacity based on sex, age and height information is determined as described in “Lung Function Testing: Selection of Reference Values and Interpretative Strategies” published in American Review Of Respiratory Disease, 144:1202-18, 1991. In one embodiment, oxygen production requirements are further calculated from the patient's breath profile. For example, the CO₂ content of exhaled breath may be monitored. Additionally or alternatively, information from the patient sensor(s) 328 is used to determine and update the ventilation of the patient. In some embodiments the determination 404 of patient's oxygen requirements comprises sending the input information to a remote system via the network 140 and receiving oxygen requirements from the remote system.

Oxygen is generated 406 as described previously. In some embodiments, oxygen generated 406 previously has been stored in the oxygen tank 242 and is retrieved when needed to ventilate a patient. The generated oxygen is mixed 408 with air. The breathing air is mixed to achieve a certain target fraction of inspired O₂ (FIO2) that is deemed to be therapeutic by the clinician. The mixed breathing air is provided 410 to the ventilation support system such that the air may be provided to the patient for ventilation.

In some embodiments, the control module 112 directs the oxygen generator 108 to generate oxygen at the concentration determined needed for the patient. This allows for use of less energy when the patient to be ventilated requires less oxygen than the maximum oxygen concentration the oxygen generator 108 is capable of generating.

### Components of the Ventilation Support System

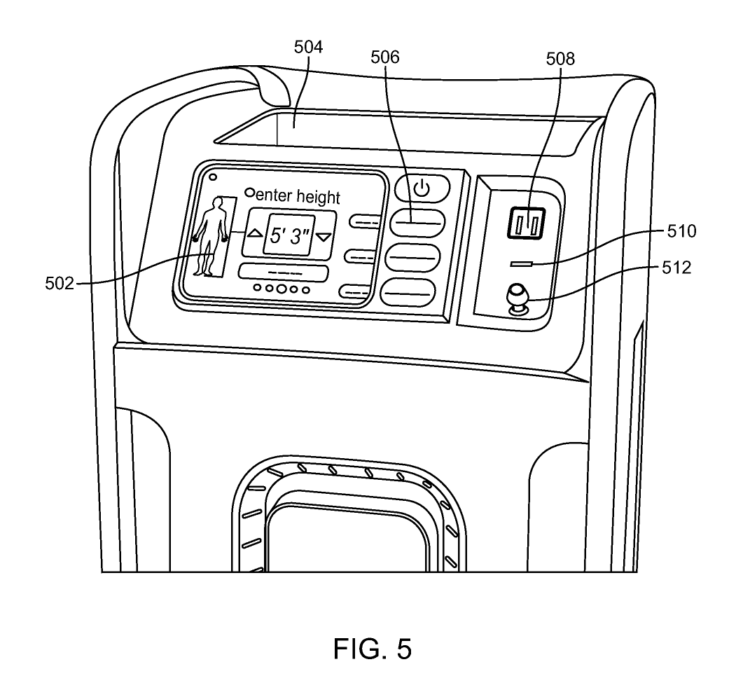

FIG. 5 is an illustration of a portion of the ventilation support system 100 including a touch screen 502, an intubation kit storage 504, multi-function buttons 506, power and data maintenance port 508, field maintenance USB port 510 and an air hookup 512. The user interface, as displayed on the touch screen 502 requests the patient's height, wherein the height information may be used to determine lung capacity and determining an appropriate amount of oxygen and breathing air to provide to a user. In the example provided, the user can enter the height of the patient by selecting up/down arrows shown on the touch screen 502 to select heights greater or less than the 5′3″ shown on the screen. The use of a patient parameter, such as height, is only an example, but a person of skill in the art would recognize that other inputs may be required and/or requested (weight, age, sex, etc.). In some embodiments, the user might be asked to enter multiple parameters about the patient, including estimated weight, age, sex, etc., and the system might collect all of these parameters to determine the appropriate amount of oxygen to provide. The interface shown in FIG. 5 also includes a variety of other buttons, including a power on/off button and various multi-function buttons 506 that can allow the user to navigate through one or more menus of the user interface (e.g., a button for moving back a screen, to the next screen, for returning to a home screen, etc.). The buttons can be designed to be easy to use and understand so that a lay person without medical training can navigate through one or more screens of the user interface.

The intubation kit storage 502 provides an area wherein a user may store tubes used to attach to a user. The power and data maintenance port 508 are used to power the ventilation support system 100. The field maintenance USB port 510 can be used to connect a storage device or any other USB enabled device to the ventilation support system 100 to store user data or to provide a control for the ventilation support unit 100. The air hookup 512 enables a controller to attach an auxiliary compressed air to the ventilation support unit 100.

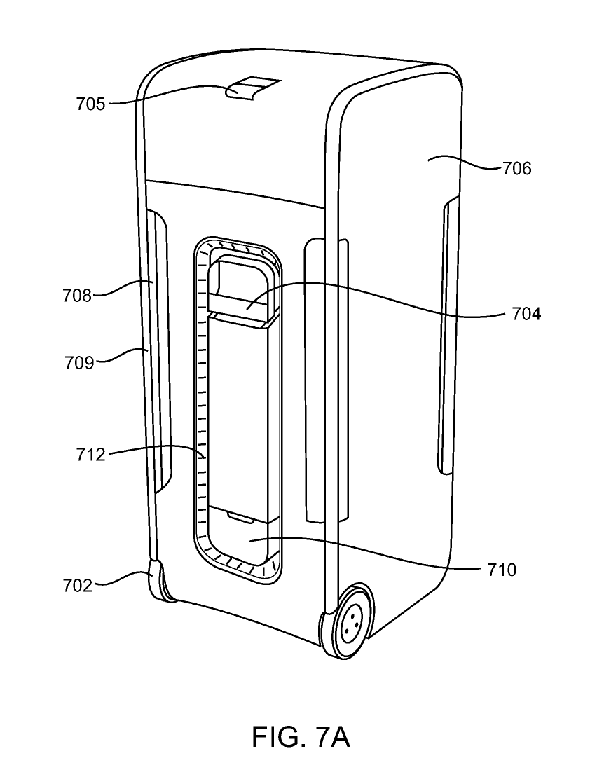

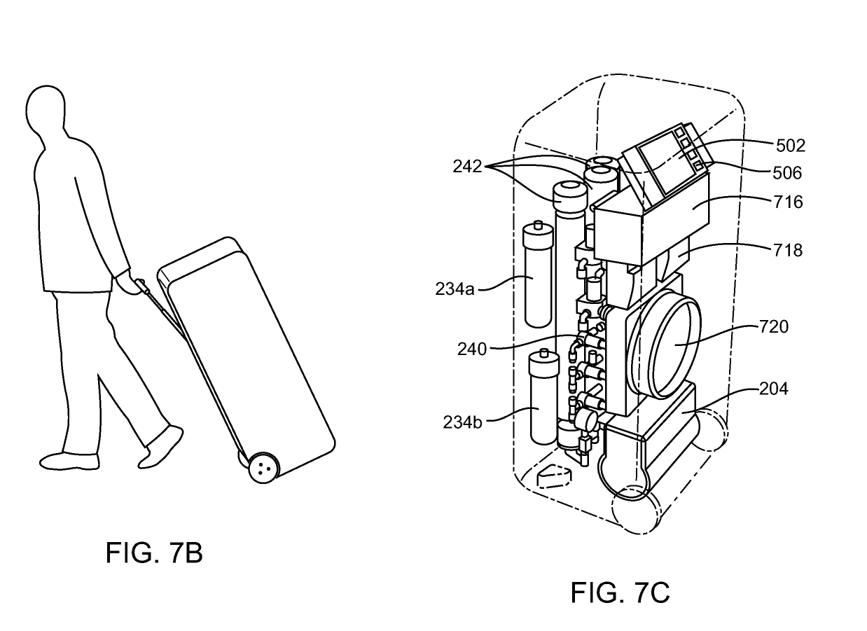

FIG. 7A illustrates a portable ventilation support system, in accordance with an embodiment of the invention. Ventilation support system 100 can be designed as a portable ventilation support system, such as that shown in FIG. 7A. It includes wheels 702 and a pull out handle 704 to enable a user to move it with relative ease. In one embodiment, the user is enabled to move the unit like a rolling suitcase. The ventilation support unit may also include a latch 705 for opening and or closing the lid. In other embodiments, other modes may be provided to enable the user to transport and move the ventilation support system. In addition, the system also includes a strong shell 706 to enable the device to withstand shock and rugged treatment. In one embodiment, a strong copolymer molded shell, similar to pelican cases may be used. In addition, the ventilation support system includes at least one reinforcing steel tube 708 to protect the device and to enable a user to handle the device and tie it down and prevent its movement. The device is also lined with rubber bumpers 709 enabling the device to absorb shock and therefore making it more rugged and durable. The rubber bumpers may be made from high durometer material in one embodiment. The device also includes a power cord 710 which is placed near the handle to enable users to quickly find it when necessary. The ventilation support system also includes vents 712 to exhaust air or exhaled breath from the device. In some embodiments, the ventilation support system 100 weighs less than 100 pounds, weighs between 50 and 70 pounds, between 30 and 70 pounds or between 30 and 50 pounds.

FIG. 7B illustrates a ventilation support system being transported by a user, in accordance with an embodiment of the invention. It illustrates a size of the ventilation support system relative to a user. In addition, the figure illustrates how a user may transport and move the ventilation support system via a handle and the wheels. As shown, the user may move the ventilation support system by tilting the unit such that it rests entirely on its wheels and pulling or pushing on the handle provided with the ventilation support system.

FIG. 7C illustrates components housed within a ventilation support system, in accordance with an embodiment of the invention. The components may include the touch screen 502, with user interface capabilities such as multi-function buttons 506, as described in reference to FIG. 5. In addition, the system may include an electronics module 716, a power supply 718 and a radiator 720. An oxygen generator may include an air compressor pump 204, one or more oxygen tanks 242, one or more molecular sieve beds 234 and check valves 240 to perform PSA and generate oxygen.

The electronics module 716 may include sensors that monitor flow, pressure and humidity as variously described in reference to FIGS. 2 and 3. The electronics module 716 also includes apparatus for communicating data over a network, the concentrator control 226 and the control module 112 as described in reference to FIGS. 1, 2 and 3. The electronics use the information entered by the user to calculate a patient's breathing needs, drive the components of the oxygen generator and the ventilator. The power supply 718 provides power to the ventilation support system 100. The power supply may provide power from batteries, from AC current or DC current. The radiator 720 transfers thermal energy from the compressed air stream to the ambient environment. This cools the air supplied to the system which improves nitrogen-absorbing capacity of the sieve beds.

The air compressor pump 204 compresses ambient air drawn by the ventilation support system and is described in further detail in reference to FIG. 2. The oxygen tanks 242 accumulates oxygen after nitrogen is adsorbed into molecular sieve beds 234 during PSA. The oxygen tanks 242 are described in greater detail in reference to FIG. 2. The molecular sieve beds 234 are described in greater detail in reference to FIG. 2. The check valves 240 regulate oxygen flowing into the oxygen tanks 242 after nitrogen is adsorbed into the sieve beds during PSA. The check valves 240 also ensure that oxygen does not flow back into the sieve beds after it has accumulated in the oxygen tanks 242.

Although the present invention has been described above with respect to several embodiments, various modifications can be made within the scope of the present invention. For example, the ventilation support system 100 can include more, fewer, or different components than those illustrated herein. Similarly, the ventilation process can include more, fewer, or different steps than those described herein, and the steps can be ordered differently. Accordingly, the disclosure of the present invention is intended to be illustrative, but not limiting, of the scope of the invention, which is set forth in the following claims.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “determining” or the like, refer to the action and processes of a computer system, or similar electronic computing device (such as a specific computing machine), that manipulates and transforms data represented as physical (electronic) quantities within the computer system memories or registers or other such information storage, transmission or display devices.

Certain aspects of the embodiments include process steps and instructions described herein in the form of an algorithm. It should be noted that the process steps and instructions of the embodiments could be embodied in software, firmware or hardware, and when embodied in software, could be downloaded to reside on and be operated from different platforms used by a variety of operating systems. The embodiments can also be in a computer program product which can be executed on a computing system.

The embodiments also relate to an apparatus for performing the operations herein. This apparatus may be specially constructed for the purposes, e.g., a specific computer, or it may comprise a general-purpose computer selectively activated or reconfigured by a computer program stored in the computer. The algorithms and displays presented herein are not inherently related to any particular computer or other apparatus. Various general-purpose systems may also be used with programs in accordance with the teachings herein, or it may prove convenient to construct more specialized apparatus to perform the method steps.

What is claimed is:

1. An apparatus for providing respiratory ventilation support to a patient, the apparatus comprising:
an oxygen generator including at least one nitrogen adsorbing molecular sieve for concentrating a supply of oxygen;
a mechanical ventilator configured to mix the oxygen supplied by the oxygen generator with ambient air drawn by the apparatus and supply the mixed oxygen and air to the patient;
a user interface configured to receive an input from an operator, the input indicating a patient size;
a processor; and
computer program code stored on a memory and configured to be executed by the processor, the computer program code including instructions for applying ventilation parameters based on the patient size;
the oxygen generator, the mechanical ventilator, the processor and the memory housed within the apparatus.

2. The apparatus of claim 1, wherein the instructions further comprise instructions for:
receiving input from the oxygen generator indicative of a current rate of oxygen generation by the oxygen generator;
receiving input from the mechanical ventilator indicative of a required rate of oxygen; and
adjusting the current rate of oxygen generation based on the required rate of oxygen.

3. The apparatus of claim 1 further comprising a carbon molecular sieve configured to store concentrated oxygen.

4. The apparatus of claim 1, wherein the nitrogen adsorbing molecular sieve includes zeolites comprising lithium.

5. The apparatus of claim 4, wherein the apparatus further comprises a vacuum apparatus configured to apply vacuum to regenerate zeolites.

6. The apparatus of claim 1, further comprising a compressor configured to compress ambient air drawn by the apparatus wherein the oxygen generator is further configured to use the compressed ambient air to concentrate the supply of oxygen, the compressor housed in the apparatus.

7. The apparatus of claim 1, further comprising a sensor module configured to monitor a differential pressure between a first pressure of the patient's lungs and a second pressure of inlet air provided by the mechanical ventilator to the patient.

8. The apparatus of claim 7, wherein:
the mixed oxygen and air has a ratio of oxygen to air; and
the instructions further comprise instructions for adjusting the ratio based on the differential pressure.

9. A method for providing respiratory ventilation support to a patient, the method comprising:
concentrating, with an oxygen generator, a supply of oxygen by removing nitrogen from ambient air drawn by a ventilation apparatus, the ventilation apparatus being portable and moveable;
receiving an input from an operator, the input indicating a patient size;
determining ventilation parameter requirements based on the patient size;
mixing the concentrated oxygen with ambient air drawn by the ventilation apparatus according to the ventilation parameters; and
supplying the mixture to a patient.

10. The method of claim 9, wherein the ventilation parameters comprise a required rate of oxygen and the method further comprising:
receiving input indicative of a current rate of oxygen generation by the oxygen generator; and
adjusting the current rate of oxygen generation based on the required rate of oxygen.

11. The method of claim 9, wherein concentrating a supply of oxygen comprises using one of a pressure swing and a vacuum swing adsorption process.

12. The method of claim 9, further comprising compressing ambient air drawn by the ventilation apparatus by a compressor housed in the ventilation apparatus.

13. The method of claim 9, further comprising storing the concentrated oxygen in a carbon molecular sieve prior to mixing with the ambient air.

14. The method of claim 9, further comprising monitoring a differential pressure between a first pressure of the patient's lungs and a second pressure of inlet air provided by the ventilation apparatus to the patient.

15. The method of claim 14, wherein mixing the concentrated oxygen with ambient air comprises mixing the concentrated oxygen with ambient air according to a ratio of oxygen to air and further comprising adjusting the ratio of oxygen to air based on the differential pressure.

16. The method of claim 9 wherein the oxygen generator concentrates the supply of oxygen at a rate of greater than 20 L/min.

17. The method of claim 9 wherein the oxygen generator concentrates the supply of oxygen at a rate of 20 L/min to 30 L/min.

18. The method of claim 9 wherein the oxygen generator concentrates the supply of oxygen at a rate of 22 L/min to 24 L/min.

19. The method of claim 9 wherein the oxygen generator concentrates the supply of oxygen to 93% oxygen or greater.

20. The method of claim 9 wherein the oxygen generator concentrates the supply of oxygen to 93% to 99% oxygen.
